(19) United States
(12) Patent Application Publication
(10) Pub. No.: US 2024/0400532 A1
(43) Pub. Date: Dec. 5, 2024

(54) NITRIC OXIDE RELEASING PRODRUGS OF MDA AND MDMA

(71) Applicant: EmpathBio, Inc., Encinitas, CA (US)

(72) Inventors: Robert B. Perni, Marlborough, MA (US); Glenn Short, Scituate, MA (US); Tanweer A. Khan, Bridgewater, NJ (US)

(21) Appl. No.: 18/426,035

(22) Filed: Jan. 29, 2024

Related U.S. Application Data
(63) Continuation of application No. 18/147,189, filed on Dec. 28, 2022, now Pat. No. 11,912,680.
(60) Provisional application No. 63/294,225, filed on Dec. 28, 2021.

(51) Int. Cl. C07D 317/58; C07D 307/79; C07D 319/18

(52) U.S. Cl. CPC C07D 317/58; C07D 307/79; C07D 319/18

(57) ABSTRACT

Provided herein are compounds of Formula (I), Formula (II), and Formula (III) or pharmaceutically acceptable salt thereof, wherein R₁, R₂, R₃, R₄, R₅, R₆, R₇ and A are defined herein. Also provided herein are pharmaceutical compositions comprising a compound of Formula (I), Formula (II), or Formula (III) and methods of using a compound of Formula (I), Formula (II), or Formula (II), e.g., in the treatment of a mental health disease or disorder.

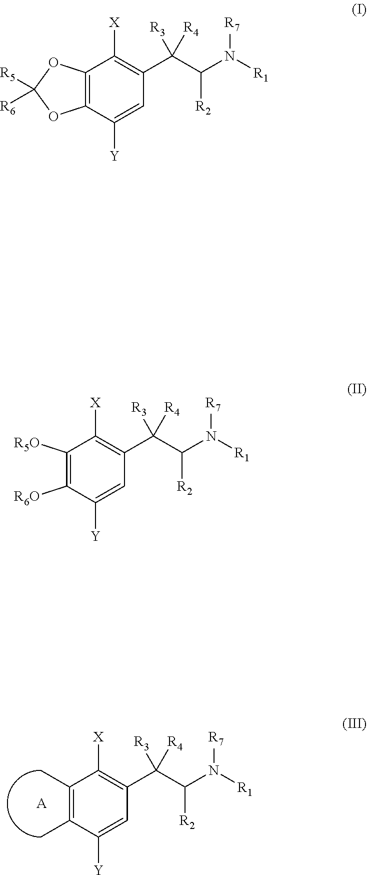

### CROSS-REFERENCE TO RELATED APPLICATION

The present application is a continuation application of U.S. application Ser. No. 18/147,189 filed Dec. 28, 2022, which claims priority to U.S. Provisional Application No. 63/294,225, filed Dec. 28, 2021, the contents of which are hereby incorporated by reference in their entirety.

### BRIEF SUMMARY OF THE INVENTION

The present disclosure provides MDA or MDMA derivatives that release nitric oxide in vivo.

The present disclosure provides prodrugs of MDMA, MDA, and derivatives thereof, as well as pharmaceutical compositions thereof.

In embodiments, the present disclosure provides a compound of Formula (I):

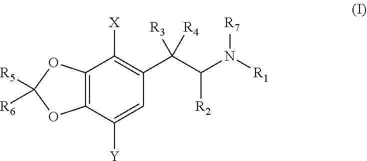

or a pharmaceutically acceptable salt thereof; wherein,
R₁ is —(C—O)(CR₉R₉′)ₙ—ONO₂ or —(C═O)(CR₉R₉′)ₘ—CH(NH₂)CH₂ONO₂;
R₂ is H, C₁-C₄ alkyl, or CF₃;
R₃ and R₄ are independently H or F;
R₅ and R₆ are independently H, C₁-C₆ alkyl, or CF₃;
R₇ is H or CH₃;
R₈ is H, C₁-C₆ alkyl, or CF₃;
R₉ and R₉′ are independently H, halogen, or C₁-C₆ alkyl;
X and Y are independently H, F, Cl, Br, or OR⁸;
n is an integer from 1 to 9; and
m is an integer from 1 to 9.

In embodiments, the present disclosure provides a compound selected from Table 1 or a pharmaceutically acceptable salt thereof.

TABLE 1
Compounds

| No. | Structure |
|---|---|
| 1-1 | 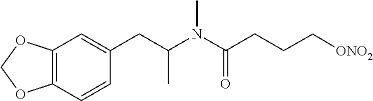 |
| 1-2 | 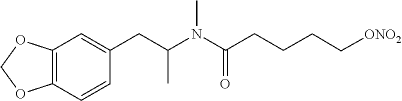 |
| 1-3 | 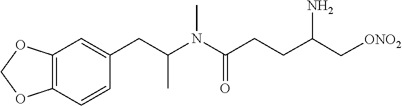 |
| 1-5 | 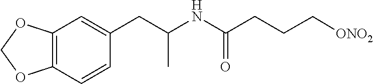 |
| 1-6 | 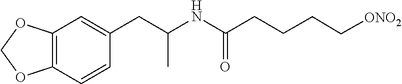 |
| 1-7 | 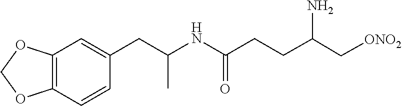 |

In embodiments, the present disclosure provides a compound of Formula (II):

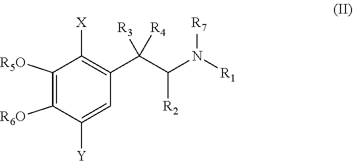

or a pharmaceutically acceptable salt thereof; wherein,
R₁ is —(C═O)(CR₉R₉′)ₘ—ONO₂ or —(C═O)(CR₉R₉′)ₘ—CH(NH₂)CH₂ONO₂;
R₂ is H, C₁-C₄ alkyl or CF₃;
R₃ and R₄ are independently H or F;
R₅ and R₆ are independently H, C₁-C₆ alkyl, or CF₃;
R₇ is H or CH₃;
R₈ is H, C₁-C₆ alkyl, or CF;
R₉ and R₉′ are independently H, halogen, or C₁-C₆ alkyl;
X and Y are independently H, F, Cl, Br, or OR⁸,
n is an integer from 1 to 9; and
m is an integer from 1 to 9.

In embodiments, the present disclosure provides a compound of Formula (III):

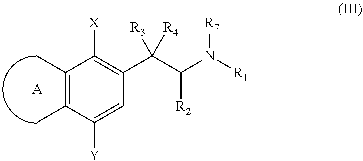

or a pharmaceutically acceptable salt thereof; wherein,
A is

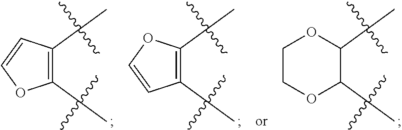 [SMILES: *c1ccoc1* ; *c1ccoc1* ; *C1OCCOC1*]

R₁ is —(C═O)(CR₉R₉′)ₙ—ONO₂ or —(C═O)(CR₉R₉′)ₘ—CH(NH₂)CH₂ONO₂,
R₂ is H, C₁-C₄ alkyl or CF₃;
R₃ and R₄ are independently H or F;
R₇ is H or CH₃;
R₈ is H, C₁-C₆ alkyl, or CF₃;
X and Y are independently H, F, Cl, Br, or OR⁸;
n is an integer from 1 to 9; and
m is an integer from 1 to 9.

In embodiments, the present disclosure provides a compound selected from Table 2 or a pharmaceutically acceptable salt thereof.

TABLE 2
Compounds

| No. | Structure |
|---|---|
| 2-1 | 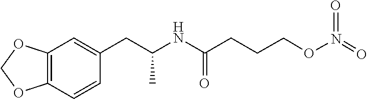 [SMILES: C[C@H](Cc1ccc2c(c1)OCO2)NC(=O)CCCO[N+](=O)[O-]] |
| 2-2 | 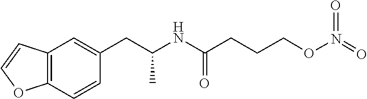 [SMILES: C[C@H](Cc1ccc2occc2c1)NC(=O)CCCO[N+](=O)[O-]] |
| 2-3 | 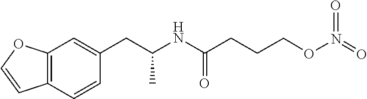 [SMILES: C[C@H](Cc1ccc2ccoc2c1)NC(=O)CCCO[N+](=O)[O-]] |
| 2-4 | 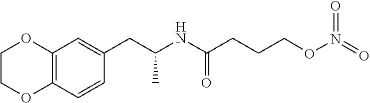 [SMILES: C[C@H](Cc1ccc2c(c1)OCCO2)NC(=O)CCCO[N+](=O)[O-]] |
| 2-5 | 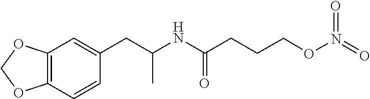 [SMILES: CC(Cc1ccc2c(c1)OCO2)NC(=O)CCCO[N+](=O)[O-]] |
| 2-6 | 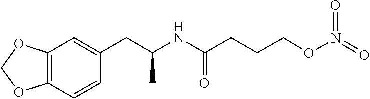 [SMILES: C[C@@H](Cc1ccc2c(c1)OCO2)NC(=O)CCCO[N+](=O)[O-]] |
| 2-7 | 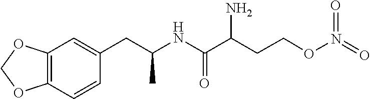 [SMILES: C[C@@H](Cc1ccc2c(c1)OCO2)NC(=O)C(N)CCO[N+](=O)[O-]] |
| 2-8 | 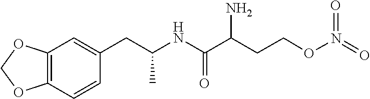 [SMILES: C[C@H](Cc1ccc2c(c1)OCO2)NC(=O)C(N)CCO[N+](=O)[O-]] |
| 2-9 | 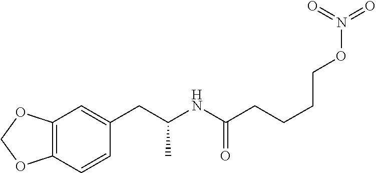 [SMILES: C[C@H](Cc1ccc2c(c1)OCO2)NC(=O)CCCCO[N+](=O)[O-]] |
| 2-10 | 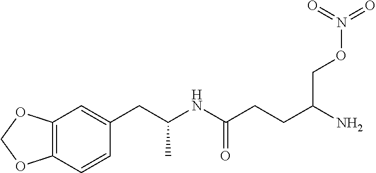 [SMILES: C[C@H](Cc1ccc2c(c1)OCO2)NC(=O)CCC(N)CO[N+](=O)[O-]] |

In some embodiments, present disclosure provides a pharmaceutical composition comprising a therapeutically effective amount of one or more compounds of the present disclosure (e.g., a compound of Formula (I), (II), (III), Table 1 or Table 2) or a pharmaceutically acceptable salt thereof and a pharmaceutically acceptable excipient.

### DETAILED DESCRIPTION OF THE INVENTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “treating” as used herein with regard to a patient or subject, refers to improving at least one symptom of the patient's or subject's disorder. In some embodiments, treating can be improving, or at least partially ameliorating a disorder or one or more symptoms of a disorder.

The term “therapeutically effective” applied to dose or amount refers to that quantity of a compound or pharmaceutical formulation that is sufficient to result in a desired clinical benefit after administration to a patient or subject in need thereof.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-cthylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₈ alkyl includes C₅ alkyls, C₄ alkyls, C₃ alkyls, C₂ alkyls and C₁ alkyl (i.e., methyl). A C₁-C₆ alkyl includes all moieties described above for C₁-C₈ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, 1-butyl, n-pentyl, 1-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkoxy” refers to a group of the formula —ORₐ where Rₐ is an alkyl, alkenyl or alknyl as defined above containing one to twelve carbon atoms. Unless stated otherwise specifically in the specification, an alkoxy group can be optionally substituted.

“Haloalkyl” refers to an alkyl, as defined above, that is substituted by one or more halo radicals, e.g., trifluoromethyl, difluoromethyl, trichloromethyl, 2,2,2-trifluorocthyl, 1,2-difluorocthyl, 3-bromo-2-fluoropropyl, 1,2-dibromoethyl, and the like. Unless stated otherwise specifically in the specification, a haloalkyl group can be optionally substituted.

The term “substituted” used herein means any of the groups described herein (e.g., alkyl, alkenyl, alkynyl, alkoxy, aryl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O) Rₕ, —NR_{g}C(═O) NR_{g}Rₕ, —NR_{g}C(═O)ORₕ, —NR_{g}SO₂Rₕ, —OC(═O) NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

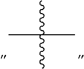

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

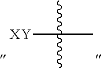

indicates that the chemical entity “XY” is bonded to another chemical entity via the point of attachment bond. Furthermore, the specific point of attachment to the non-depicted chemical entity can be specified by inference. For example, the compound CH₃-R^{3X}, wherein R^{3X} is H or

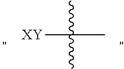

infers that when R^{3X} is “XY”, the point of attachment bond is the same bond as the bond by which R^{3X} is depicted as being bonded to CH₃.

### Compounds

In one aspect, the present disclosure provides prodrugs of MDMA, MDA, and derivatives thereof, that release nitric oxide in vivo.

In embodiments, the present disclosure provides a compound of Formula (I):

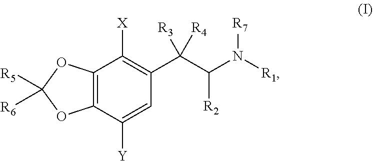

or a pharmaceutically acceptable salt thereof; wherein,
R₁ is —(C═O)(CR₉R₉′)ₙ—ONO₂ or —(C═O)(CR₉R₉′)ₘ—CH(NH₂)CH₂ONO₂;
R₂ is H, C₁-C₄ alkyl, or CF₃;
R₃ and R₄ are independently H or F;
R₅ and R₆ are independently H, C₁-C₆ alkyl, or CF₃;
R₇ is H or CH₃; and
R₈ is H, C₁-C₆ alkyl, or CF₃;
R₉ and R₉′ are independently H, halogen, or C₁-C₆ alkyl;
X and Y are independently H, F, Cl, Br, or OR⁸;
n is an integer from 1 to 9; and
m is an integer from 1 to 9.

In embodiments of the compounds of Formula (I), R₁ is —(C═O)(CH₂)ₙ—ONO₂.

In embodiments of the compounds of Formula (I), R₁ is —(C═O)(CH₂)ₘ—CH(NH₂)CH₂ONO₂.

In embodiments of the compounds of Formula (I), R₂ is C₁-C₄ alkyl.

In embodiments of the compounds of Formula (I), R₂ is methyl.

In embodiments of the compounds of Formula (I), R₂ is CF₃.

In embodiments of the compounds of Formula (I), R₃ and R₄ are H.

In embodiments of the compounds of Formula (I), R₃ and R₄ are F.

In embodiments of the compounds of Formula (I), R₅ and R₆ are H.

In embodiments of the compounds of Formula (I), R₅ and R₆ are C₁-C₆ alkyl.

In embodiments of the compounds of Formula (I), R₅ and R₆ are CF₃.

In embodiments of the compounds of Formula (I), R₇ is H.

In embodiments of the compounds of Formula (I), R₇ is CH₃.

In embodiments of the compounds of Formula (I), X and Y are H.

In embodiments of the compounds of Formula (I), X and Y are F.

In embodiments of the compounds of Formula (I), m is 2.

In embodiments of the compounds of Formula (I), n is 3.

In embodiments of the compounds of Formula (I), n is 4.

In embodiments, the present disclosure provides a compound selected from Table 1 or a pharmaceutically acceptable salt thereof.

TABLE 1
Compounds

| No. | Structure |
|---|---|
| 1-1 | 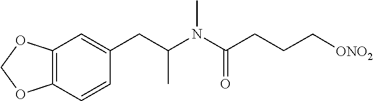 |
| 1-2 | 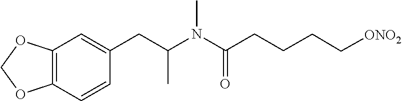 |
| 1-3 | 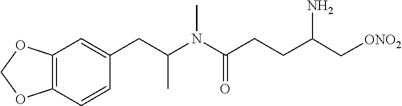 |
| 1-5 | 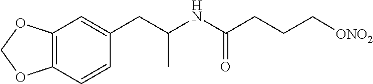 |
| 1-6 | 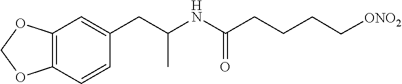 |
| 1-7 | 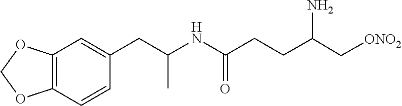 |

In embodiments, the present disclosure provides a compound of Formula (II):

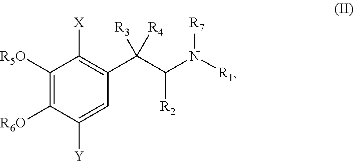

or a pharmaceutically acceptable salt thereof; wherein,
R₁ is —(C═O)(CR₉R₉′)ₙ—ONO₂ or —(C═O)(CR₉R₉′)ₘ—CH(NH₂)CH₂ONO₂;
R₂ is H, C₁-C₄ alkyl, or CF₃;
R₃ and R₄ are independently H or F;
R₅ and R₆ are independently H, C₁-C₆ alkyl, or CF₃;
R₇ is H or CH₃;
R₈ is H, C₁-C₆ alkyl, or CF₃;
R₉ and R₉′ are independently H, halogen, or C₁-C₆ alkyl;
X and Y are independently H, F, Cl, Br, or OR⁸;
n is an integer from 1 to 9; and
m is an integer from 1 to 9.

In embodiments, the present disclosure provides a compound of Formula (III):

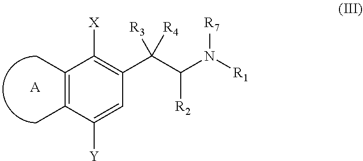

or a pharmaceutically acceptable salt thereof; wherein,
A is

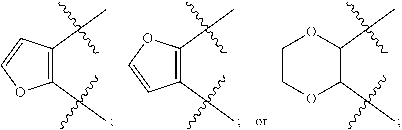 [SMILES: *c1ccoc1* ; *c1ccoc1* ; *C1OCCOC1*]

R₁ is —(C═O)(CR₉R₉′)ₙ—ONO₂ or —(C═O)(CR₉R₉′)ₘ—CH(NH₂)CH₂ONO₂;
R₂ is H, C₁-C₄ alkyl or CF₃;
R₃ and R₄ are independently H or F;
R₅ and R₆ are independently H, C₁-C₆ alkyl, or CF₃;
R₇ is H or CH₃;
R₈ is H, C₁-C₆ alkyl, or CF₃;
R₉ and R₉′ are independently H, halogen, or C₁-C₆ alkyl;
X and Y are independently H, F, Cl, Br, or OR⁸;
n is an integer from 1 to 9; and
m is an integer from 1 to 9.

In embodiments of the compounds of Formula (III), A is

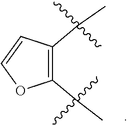 [SMILES: *c1ccoc1*]

In embodiments of the compounds of Formula (III), A is

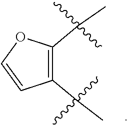 [SMILES: *c1ccoc1*]

In embodiments of the compounds of Formula (III), A is

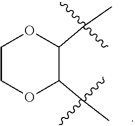 [SMILES: *C1OCCOC1*]

In embodiments of the compounds of Formula (II) or Formula (III), R₁ is —(C═O)(CR₉R₉′)ₙ—ONO₂.

In embodiments of the compounds of Formula (II) or Formula (III), R₁ is —(C═O)(CR₉R₉′)ₘ—CH(NH₂)CH₂ONO₂.

In embodiments of the compounds of Formula (II) or Formula (III), R₉ and R₉′ are independently halogen or C₁-C₆ alkyl.

In embodiments of the compounds of Formula (II) or Formula (III), R₁ is —(C═O)(CH₂)ₙ—ONO₂.

In embodiments of the compounds of Formula (II) or Formula (III), R₁ is —(C═O)(CH₂)ₘ—CH(NH₂)CH₂ONO₂.

In embodiments of the compounds of Formula (II) or Formula (III), R₂ is C₁-C₄ alkyl.

In embodiments of the compounds of Formula (II) or Formula (III), R₂ is methyl.

In embodiments of the compounds of Formula (II) or Formula (III), R₂ is CF₃.

In embodiments of the compounds of Formula (II) or Formula (III), R₃ and R₄ are H.

In embodiments of the compounds of Formula (II) or Formula (III), R₃ and R₄ are F.

In embodiments of the compounds of Formula (II), R₅ and R₆ are H.

In embodiments of the compounds of Formula (II), R₅ and R₆ are C₁-C₆ alkyl.

In embodiments of the compounds of Formula (II), R₅ and R₆ are CF₃.

In embodiments of the compounds of Formula (II) or Formula (III), R₇ is H.

In embodiments of the compounds of Formula (II) or Formula (III), R₇ is CH₃.

In embodiments of the compounds of Formula (II) or Formula (III), X and Y are H.

In embodiments of the compounds of Formula (II) or Formula (III), X and Y are F.

In embodiments of the compounds of Formula (II) or Formula (III), m is 2.

In embodiments of the compounds of Formula (II) or Formula (III), n is 3.

In embodiments of the compounds of Formula (II) or Formula (III), n is 4.

In embodiments, the present disclosure provides a compound selected from Table 2 or a pharmaceutically acceptable salt thereof.

TABLE 2
Compounds

| No. | Structure | Name |
|---|---|---|
| 2-1 | 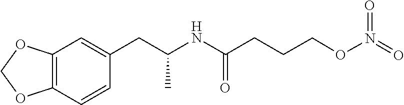 [SMILES: C[C@H](Cc1ccc2c(c1)OCO2)NC(=O)CCCO[N+](=O)[O-]] | (R)-4-((1-(benzo[d][1,3]dioxol-5- yl)propan-2-yl)amino)-4-oxobutyl nitrate |
| 2-2 | 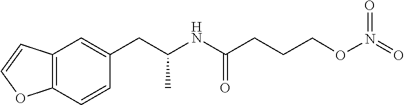 [SMILES: C[C@H](Cc1ccc2occc2c1)NC(=O)CCCO[N+](=O)[O-]] | (R)-4-((1-(benzofuran-5-yl)propan- 2-yl)amino)-4-oxobutyl nitrate |
| 2-3 | 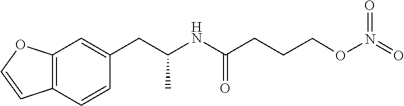 [SMILES: C[C@H](Cc1ccc2ccoc2c1)NC(=O)CCCO[N+](=O)[O-]] | (R)-4-((1-(benzofuran-6-yl)propan- 2-yl)amino)-4-oxobutyl nitrate |
| 2-4 | 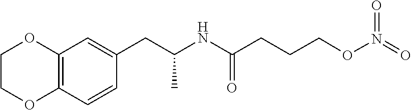 [SMILES: C[C@H](Cc1ccc2c(c1)OCCO2)NC(=O)CCCO[N+](=O)[O-]] | (R)-4-((1-(2,3- dihydrobenzo[b][1,4]dioxin-6- yl)propan-2-yl)amino)-4-oxobutyl nitrate |
| 2-5 | 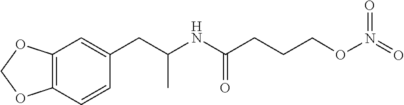 [SMILES: CC(Cc1ccc2c(c1)OCO2)NC(=O)CCCO[N+](=O)[O-]] | 4-((1-(benzo[d][1,3]dioxol-5- yl)propan-2-yl)amino)-4-oxobutyl nitrate |
| 2-6 | 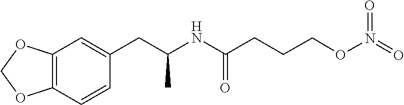 [SMILES: C[C@@H](Cc1ccc2c(c1)OCO2)NC(=O)CCCO[N+](=O)[O-]] | (S)-4-((1-(benzo[d][1,3]dioxol-5- yl)propan-2-yl)amino)-4-oxobutyl nitrate |
| 2-7 | 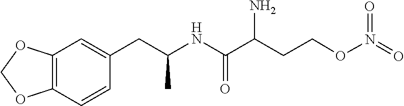 [SMILES: C[C@@H](Cc1ccc2c(c1)OCO2)NC(=O)C(N)CCO[N+](=O)[O-]] | 3-amino-4-(((S)-1- (benzo[d][1,3]dioxol-5-yl)propan- 2-yl)amino)-4-oxobutyl nitrate |
| 2-8 | 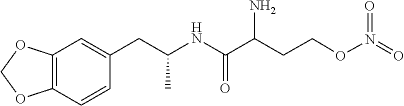 [SMILES: C[C@H](Cc1ccc2c(c1)OCO2)NC(=O)C(N)CCO[N+](=O)[O-]] | 3-amino-4-(((R)-1- (benzo[d][1,3]dioxol-5-yl)propan- 2-yl)amino)-4-oxobutyl nitrate |
| 2-9 | 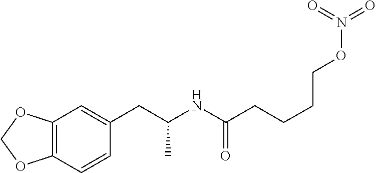 [SMILES: C[C@H](Cc1ccc2c(c1)OCO2)NC(=O)CCCCO[N+](=O)[O-]] | (R)-5-((1-(benzo[d][1,3]dioxol-5- yl)propan-2-yl)amino)-5-oxopentyl nitrate |
| 2-10 | 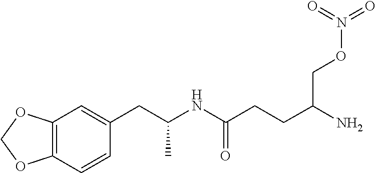 [SMILES: C[C@H](Cc1ccc2c(c1)OCO2)NC(=O)CCC(N)CO[N+](=O)[O-]] | 2-amino-5-(((R)-1- (benzo[d][1,3]dioxol-5-yl)propan- 2-yl)amino)-5-oxopentyl nitrate |

### Compositions

In one aspect, the present disclosure provides a pharmaceutical composition comprising a therapeutically effective amount of one or more compounds of the present disclosure (e.g., a compound of Formula (I), (II), (III), Table 1 or Table 2) or a pharmaceutically acceptable salt thereof and a pharmaceutically acceptable excipient.

The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

### Methods of Treatment

In embodiments, the present disclosure provides a method of treating or preventing neurological disorders in a subject in need thereof, the method comprising administering to the subject a therapeutically effective amount of a compound disclosed herein, e.g., Formula (I), (II), (III), Table 1 or Table 2, or a pharmaceutically acceptable salt thereof.

In embodiments, the neurological disorder is a mood disorder. In embodiments, the mood disorder is clinical depression, postnatal depression or postpartum depression, perinatal depression, atypical depression, melancholic depression, psychotic major depression, cationic depression, seasonal affective disorder, dysthymia, double depression, depressive personality disorder, recurrent brief depression, major depressive disorder, minor depressive disorder, bipolar disorder or manic depressive disorder, depression caused by chronic medical conditions, treatment-resistant depression, refractory depression, suicidality, suicidal ideation, or suicidal behavior. In embodiments, the method described herein provides therapeutic effect to a subject suffering from depression (e.g., moderate or severe depression). In some embodiments, the mood disorder is associated with neuroendocrine diseases and disorders, neurodegenerative diseases and disorders (e.g., epilepsy), movement disorders, tremor (e.g., Parkinson's Disease), or women's health disorders or conditions. In embodiments the mood disorder is depression. In embodiments, the mood disorder is treatment-resistant depression or major depressive disorder. In some embodiments, the mood disorder is major depressive disorder. In embodiments, the mood disorder is treatment-resistant depression.

In some embodiments, the present disclosure provides a method of treating or preventing PTSD, mood disorders, general anxiety disorder, addictive disorders, and/or drug dependence in a subject in need thereof, the method comprising administering to the subject a therapeutically effective amount of a compound disclosed herein, e.g., a compound of Formula (I), (II), (III), Table 1, or Table 2, a pharmaceutically acceptable salt thereof, or a pharmaceutical composition thereof.

In embodiments, the compounds of the present disclosure are used to treat PTSD. In embodiments, the compounds of the present disclosure are used for induction and maintenance therapy to treat PTSD. In embodiments, the compounds of the present disclosure are used to treat PTSD with an improved safety profile when compared to treatment with the entactogenic, oneirophrenic or psychedelic compound (e.g. MDMA or related compound, psilocybin or dimethyltryptamine) alone. In embodiments, the compounds of the present disclosure are used for induction and maintenance therapy to treat PTSD with an improved safety profile when compared to treatment with the entactogenic, oneirophrenic or psychedelic compound (e.g. MDMA or related compound, psilocybin or dimethyltryptamine) alone.

In embodiments, the compounds of the present disclosure are used to treat behavioral or mood disorders. Examples of behavioral or mood disorders include anxiety, such as social anxiety in autistic subjects (e.g. autistic adults) and anxiety related to life-threatening illnesses, stress (where moderation thereof is measured, for example, by effects on amygdala responses). In some embodiments, the anxiety disorder is panic disorder, obsessive-compulsive disorder, or general anxiety disorder. Other examples include lack of motivation, attention, accuracy, speed of response, perseveration, and/or cognitive engagement. Further examples include depression (e.g., MDD or TRD), attention disorders, disorders of executive function and/or cognitive engagement, obsessive compulsive disorder, bipolar disorder, panic disorder, phobia, schizophrenia, psychopathy, antisocial personality disorder and/or neurocognitive disorders.

In embodiments, the compounds the present disclosure are used to treat an addictive disorder. In embodiments, the addictive disorder is alcohol abuse, substance abuse, smoking, or obesity. In embodiments, the disorder is an eating disorder (anorexia nervosa, bulimia, nervosa, binge eating disorder, etc.) or an auditory disorder.

In embodiments, the disorder is an impulsive disorder. In embodiments, the impulsive disorder is attention deficit hyperactivity disorder (ADHD), attention deficit disorder (ADD), Tourette's syndrome or autism.

In embodiments, the disorder is a compulsive disorder. In embodiments, the compulsive disorder is obsessive compulsive disorder (OCD), gambling, or aberrant sexual behavior.

In embodiments, the disorder is a personality disorder. In embodiments, the personality disorder is conduct disorder, antisocial personality, or aggressive behavior.

### Numbered Embodiments

In addition to the disclosure above, the Examples below, and the appended claims, the disclosure sets for the following numbered embodiments.

1. A of Formula (I):

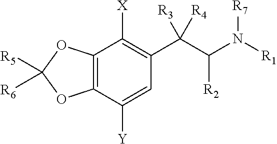

or a pharmaceutically acceptable salt thereof; wherein,
R₁ is —(C═O)(CR₉R₉′)ₙ—ONO₂ or —(C—O)(CR₉R₉′)ₘ—CH(NH₂)CH₂ONO₂;
R₂ is H, C₁-C₄ alkyl, or CF₃;
R₃ and R₄ are independently H or F;
R₅ and R₆ are independently H, C₁-C₆ alkyl, or CF₃;
R₇ is H or CH₃;
R₈ is H, C₁-C₆ alkyl, or CF₃;
R₉ and R₉′ are independently H, halogen, or C₁-C₆ alkyl;
X and Y are independently H, F, Cl, Br, or OR⁸,
n is an integer from 1 to 9; and
m is an integer from 1 to 9.

2. The compound of embodiment 1, wherein R₁ is —(C═O)(CR₉R₉′)ₙ—ONO₂.

3. The compound of embodiment 1, wherein R₁ is —(C═O)(CR₉R₉′)ₘ—CH(NH₂)CH₂ONO₂.

3a. The compound of embodiment 1, wherein R₁ is —(C═O)(CH₂)ₙ—ONO₂.

4. The compound of embodiment 1, wherein R₁ is —(C═O)(CH₂)ₘ—CH(NH₂)CH₂ONO₂.

5. The compound of any one of embodiments 1-4, wherein R₂ is C₁-C₄ alkyl.

6. The compound of any one of embodiments 1-4, wherein R₂ is methyl.

7. The compound of any one of embodiments 1-4, wherein R₂ is CF₃.

8. The compound of any one of embodiments 1-7, wherein R₃ and R₄ are H.

9. The compound of any one of embodiments 1-7, wherein R₃ and R₄ are F.

10. The compound of any one of embodiments 1-9, wherein R₅ and R₆ are H.

11. The compound of any one of embodiments 1-9, wherein R₅ and R₆ are C₁-C₆ alkyl.

12. The compound of any one of embodiments 1-9, wherein R₅ and R₆ are CF₃.

13. The compound of any one of embodiments 1-12, wherein R₇ is H.

14. The compound of any one of embodiments 1-12, wherein R₇ is CH₃.

15. The compound of any one of embodiments 1-14, wherein X and Y are H.

16. The compound of any one of embodiments 1-14, wherein X and Y are F.

17. The compound of any one of embodiments 1-16, wherein m is 2.

18. The compound of any one of embodiments 1-17, wherein n is 3.

19. The compound of any one of embodiments 1-17, wherein n is 4.

20. The compound of embodiment 1, having the formula:

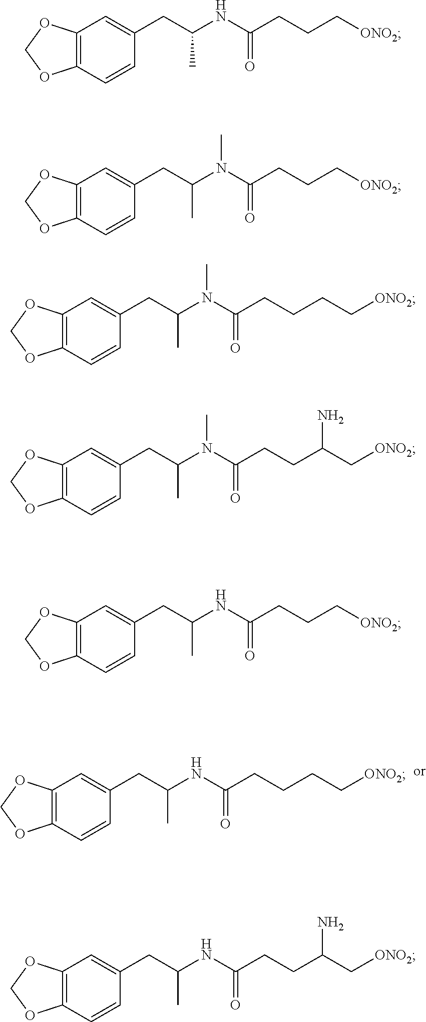

or a pharmaceutically acceptable salt thereof.

21. A compound of Formula (II):

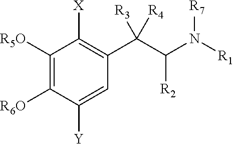

or a pharmaceutically acceptable salt thereof; wherein,
R₁ is —(C═O)(CR₉R₉′)ₙ—ONO₂ or —(C═O)(CR₉R₉′)ₘ—CH(NH₂)CH₂ONO₂;
R₂ is H, C₁-C₄ alkyl or CF₃;
R₃ and R₄ are independently H or F;
R₅ and R₆ are independently H, C₁-C₆ alkyl, or CF₃;
R₇ is H or CH₃;
R₈ is H, C₁-C₆ alkyl, or CF;
R₉ and R₉′ are independently H, halogen, or C₁-C₆ alkyl;
X and Y are independently H, F, Cl, Br, or OR⁸,
n is an integer from 1 to 9; and
m is an integer from 1 to 9.

22. The compound of embodiment 21, wherein R₁ is —(C═O)(CR₉R₉′)ₙ—ONO₂.

23. The compound of embodiment 21, wherein R₁ is —(C═O)(CR₉R₉′)ₘ—CH(NH₂)CH₂ONO₂.

24. The compound of embodiment 21, wherein R₁ is —(C═O)(CH₂)ₙ—ONO₂.

25. The compound of embodiment 21, wherein R₁ is —(C═O)(CH₂)ₘ—CH(NH₂)CH₂ONO₂.

26. The compound of any one of embodiments 21-25, wherein R₂ is C₁-C₄ alkyl.

27. The compound of any one of embodiments 21-26, wherein R₂ is methyl.

28. The compound of any one of embodiments 21-25, wherein R₂ is CF₃.

29. The compound of any one of embodiments 21-28, wherein R₃ and R₄ are H.

30. The compound of any one of embodiments 21-28, wherein R₃ and R₄ are F.

31. The compound of any one of embodiments 21-30, wherein R₅ and R₆ are H.

32. The compound of any one of embodiments 21-30, wherein R₅ and R₆ are C₁-C₆ alkyl.

33. The compound of any one of embodiments 21-30, wherein R₅ and R₆ are CF₃.

34. The compound of any one of embodiments 21-33, wherein R₇ is H.

35. The compound of any one of embodiments 21-33, wherein R₇ is CH₃.

36. The compound of any one of embodiments 21-35, wherein X and Y are H.

37. The compound of any one of embodiments 21-35, wherein X and Y are F.

38. The compound of any one of embodiments 21-37, wherein m is 2.

39. The compound of any one of embodiments 21-38, wherein n is 3.

40. The compound of any one of embodiments 21-38, wherein n is 4.

41. A compound of Formula (III):

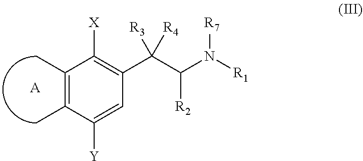

or a pharmaceutically acceptable salt thereof; wherein,
A is

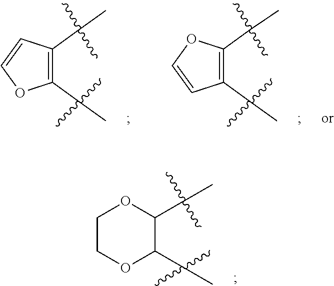 [SMILES: *c1ccoc1* ; *c1ccoc1* ; *C1OCCOC1*]

R₁ is —(C═O)(CR₉R₉′)ₙ—ONO₂ or —(C═O)(CR₉R₉′)ₘ—CH(NH₂)CH₂ONO₂,
R₂ is H, C₁-C₄ alkyl or CF₃;
R₃ and R₄ are independently H or F;
R₅ and R₆ are independently H, C₁-C₆ alkyl, or CF₃;
R₇ is H or CH₃;
R₈ is H, C₁-C₆ alkyl, or CF₃;
X and Y are independently H, F, Cl, Br, or OR⁸,
n is an integer from 1 to 9; and
m is an integer from 1 to 9.

42. The compound of embodiment 41, wherein R₁ is —(C═O)(CR₉R₉′)ₙ—ONO₂.

43. The compound of embodiment 41, wherein R₁ is —(C═O)(CR₉R₉′)ₘ—CH(NH₂)CH₂ONO₂.

44. The compound of embodiment 41, wherein R₁ is —(C═O)(CH₂)ₙ—ONO₂.

45. The compound of embodiment 41, wherein R₁ is —(C═O)(CH₂)ₘ—CH(NH₂)CH₂ONO₂.

46. The compound of any one of embodiments 41-45, wherein R₂ is C₁-C₄ alkyl.

47. The compound of any one of embodiments 41-46, wherein R₂ is methyl.

48. The compound of any one of embodiments 41-45, wherein R₂ is CF₃.

49. The compound of any one of embodiments 41-48, wherein R₃ and R₄ are H.

50. The compound of any one of embodiments 41-48, wherein R₃ and R₄ are F.

51. The compound of any one of embodiments 41-50, wherein R₇ is H.

52. The compound of any one of embodiments 41-50, wherein R₇ is CH₃.

53. The compound of any one of embodiments 41-52, wherein X and Y are H.

54. The compound of any one of embodiments 41-52, wherein X and Y are F.

55. The compound of any one of embodiments 41-54, wherein m is 2.

56. The compound of any one of embodiments 41-55, wherein n is 3.

57. The compound of any one of embodiments 41-55, wherein n is 4.

58. The compound of any one of embodiments 41-57, wherein A is

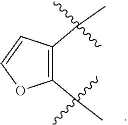 [SMILES: *c1ccoc1*]

59. The compound of any one of embodiments 41-57, wherein A is

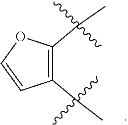 [SMILES: *c1ccoc1*]

60. The compound of any one of embodiments 41-57, wherein A is

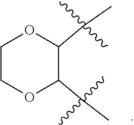 [SMILES: *C1OCCOC1*]

61. A compound having the formula:

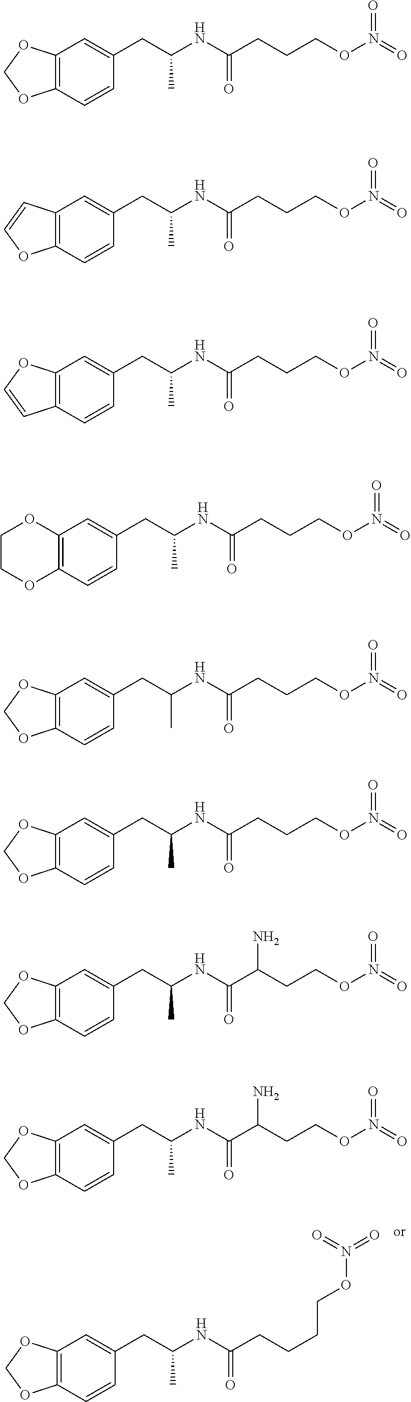

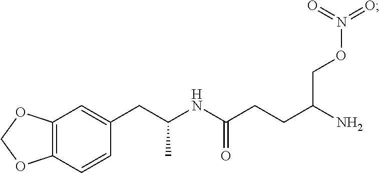 [SMILES: C[C@H](Cc1ccc2c(c1)OCO2)NC(=O)CCC(N)CO[N+](=O)[O-]]

or a pharmaceutically acceptable salt thereof.

62. A pharmaceutical composition, comprising a compound of any one of embodiments 1-61 and a pharmaceutically acceptable excipient.

63. A method of treating a mental health disease or disorder, the method comprising administering a therapeutically effective amount of a compound of any one of embodiments 1-61 or the pharmaceutical composition of embodiment 62.

### EXAMPLES

### Example 1: Methods of Preparing the Compounds of the Present Disclosure

The following schemes provide methods for preparing the compounds of the present disclosure.

Unless otherwise noted, all materials/reagents were obtained from commercial suppliers and used without further purification. Reactions were monitored by LC-MS and/or thin layer chromatography (TLC) on silica gel 60 F254 (0.2 mm) pre-coated aluminum foil or glass-backed and visualized using UV light. ¹HNMR (400 MHZ) spectra was recorded on Broker spectrometers at RT with TMS or the residual solvent peak as the internal standard. Chemical shifts are given in (δ) and the coupling constants (J) are given as absolute values in Hertz (Hz). The multiplicities in ¹HNMR spectra are abbreviated as follows: s (singlet), d (doublet), t (triplet), q (quartet), m (multiplet), br or broad (broadened). Preparative HPLC purifications were performed on Shimadzu LC-6AD. All purification work was completed using a Shim-pack PREP-DDS (H) KIT Column. The mobile phases were water (with 0.1% HCO₂H) and acetonitrile; all reagents used were of HPLC grade. The flow rate was 10 ml/min. LC-MS analyses were performed on Shimadzu LCMS-2020 equipped with LC-20AD or 30AD pumps, SPD-M20A PDA and Alltech 3300 ELSD; Mobile Phase: A: Water (0.1% Formic acid), B: ACN; 5 minute run; Column: Sepax BR-C18 4.6*50 mm,3 μm; Flow Rate: 1.0 ml/min; Oven Temperature: 40° C.; Gradient: 20% B for 0.2 min, increase to 70% B within 1.8 min, 70% B for 2.8 min, back to 20% B within 0.2 min, 20% B for 2 min). Preparative TLC was performed on Whatman LK6F Silica Gel 60A size 20×20 cm plates with a thickness of 1000 μm or equivalent.

### Method a for Preparing Compounds of the Present Disclosure, e.g., Formula 1-1, 1-2,1-3, 1-5, 1-6, 1-7 and 1-8

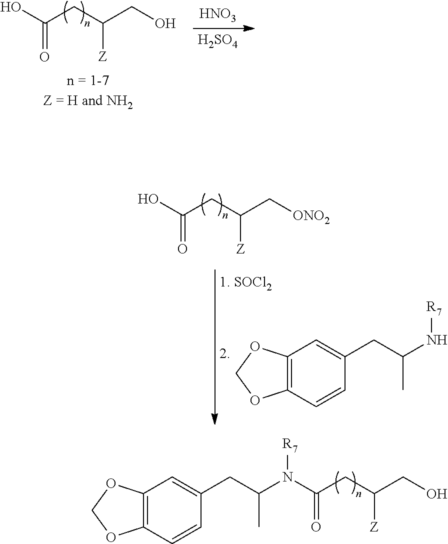

### Example 2: Synthesis of 2-1: (R)-4-((1-(Benzo[d][1,3]Dioxol-5-Yl) Propan-2-Yl)Amino)-4-Oxobutyl Nitrate

Intermediate 2-14 was synthesized from commercially available intermediate 2-1-A (Scheme 1) and was used for the final pro drig synthesis (Scheme 2).

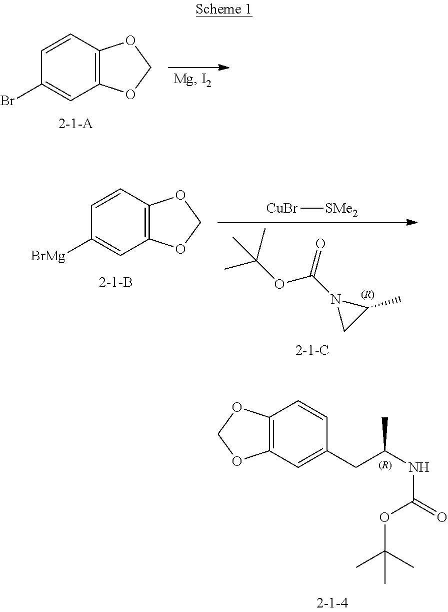

### Synthesis of Intermediate 2-1-B

To a 100 mL flask was charged magnesium (60.4 mg, 2.487 mmol, 2.00 equiv.), iodine (one crystal) and tetrahydrofuran (1.5 mL) at room temperature under nitrogen. 5-Bromo-2H-1,3-benzodioxole, 2-1-A (50 mg) was added to the mixture and heated to 50° C. at which time the iodine color disappeared, and the internal temperature rose to 56° C. 5-Bromo-2H-1,3-benzodioxole (450 mg, total added 500 mg, 2.487 mmol, 2.0 equiv.) was added, via syringe, to the mixture dropwise maintaining an internal temperature of 45-55° over 10 minutes. After addition was complete the syringe was rinsed with THF (0.1 mL) and the rinse charged to the reaction at 49° C. After stirring for 1.5 hours the batch was a clear amber color with an internal temperature of 19.6° C. THF (1 mL) was added. This crude material 2-1-B was used directly for the next steps.

### Synthesis of Intermediate 2-1-4

The flask was cooled to 0.8° C. using an ice/water bath then solid CuBr•SMe2 (52.3 mg, 0.254 mmol, 0.2 equiv) was charged in one portion. An exotherm to 6° C. was observed. After cooling to 0.5° C. a solution of tert-butyl (2R)-2-methylaziridine-1-carboxylate, 2-1-C(200 mg, 1.272 mmol, 1.0 equiv.) in tetrahydrofuran (0.5 mL) was added over 20 minutes, while maintaining an internal temperature <6° C. After stirring for 4 hours TLC analysis (5:1 heptane/EA) of the brown slurry showed complete reaction. After a further 20 minutes the reaction was quenched with dropwise addition sat. ammonium chloride (5.0 mL), while maintaining an internal temperature <18° C. (3 minutes). After stirring for 12 minutes at room temperature the biphasic mixture was diluted with Ethyl acetate (1.5 mL). The layers were separated, and the aqueous layer was extracted with Ethyl acetate (2×1.5 mL). The combined organic layers dried over sodium sulfate (1.3 g), filtered, and concentrated under reduced pressure. Chromatographic purification in silica, eluting with 0-15% Ethyl acetate/heptane afforded tert-butyl (R)-(1-(benzo[d][1,3]dioxol-5-yl) propan-2-yl) carbamate, 2-1-4 (130 mg, 36% yield) as a white solid. 50 mg purified by reversed Flash with the following conditions: Column: XBridge Prep OBD C18 Column, 30*150 mm, 5 μm; Mobile Phase A: 10 mmol NH₄HCO₃+0.05% NH₃H₂O, Mobile Phase B: ACN; Flow rate: 60 mL/min; Gradient: 23% B to 53% B in 8 min, 53% B; Wavelength: 254 nm. The fractions of desired product were lyophilized. This resulted in 28.0 mg as a white solid. MS m/z [M−H]-(ESI): 278.15. ¹H NMR (400 MHZ, DMSO-d₆) δ 6.81-6.61 (m, 4H), 5.95-5.94 (m, 2H), 3.61-3.53 (m, 1H), 2.63-2.58 (m, 1H), 2.49-2.44 (m, 1H), 1.34 (s, 9H), 0.98 (d, J=6.8 Hz, 3H).

Synthesis of intermediate 2-1: tert-butyl (R)-(1-(benzo[d][1,3]dioxol-5-yl) propan-2-yl) carbamate

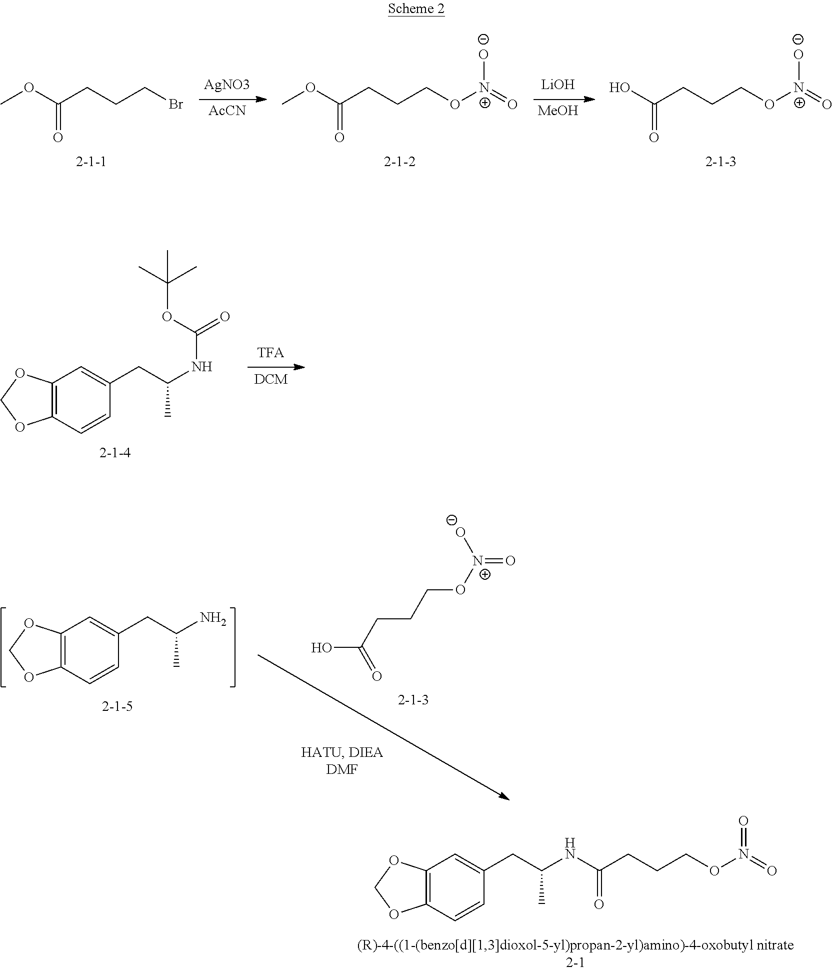

### Synthesis of Intermediate 2-1-2

To a solution of methyl 4-bromobutanoate, 2-1-1 (1 g, 5.56 mmol, 1 equiv.) in dry acetonitrile (20 mL) was added silver nitrate (2.35 g, 13.810 mmol, 2.5 equiv.). The reaction was heated to 80° C. for 4 hours, protected from light. The reaction mixture was filtered through Celite and the solvent removed under reduced pressure. The crude residue suspended in ethyl acetate (20 mL) and filtered through a silica plug. The organic layer was washed with water (10 mL) and brine (10 mL), dried over sodium sulfate, filtered and the solvent removed under reduced pressure to 2-1-2 (800 mg, 89%) as a pale yellow oil, which was used without any further purification.

### Synthesis of Intermediate 2-1-3:4-(Nitrooxy) Butanoic Acid

Methyl 4-(nitrooxy) butanoate 2-1-1 (700 mg, 4.291 mmol, 1 equiv) was dissolved in methanol (16.8 mL) at 5° C. and LiOH (4.2 mL, 2 mol/L in water) was added. The reaction was stirred overnight at 5° C. Solution was acidified to pH 3 with HCl (1 mol/L) and methanol as evaporated. Aqueous phase was extracted with DCM, dried over sodium sulfate and concentrated affording 4-(nitrooxy) butanoic acid, 2-1-3 (350 mg, 55%) as a yellow oil.

### Synthesis of Final Product 2-1: (R)-4-((1-(Benzo[d][1,3]Dioxol-5-Yl) Propan-2-Yl)Amino)-4-Oxobutyl Nitrate

To a solution of 2-1-4, tert-butyl (R)-(1-(benzo[d][1,3]dioxol-5-yl) propan-2-yl) carbamate (200 mg, 0.716 mmol, 1 equiv) in dichloromethane (3.3 mL), TFA (0.7 mL) was added. It was stirred for one hour and concentrated under reduced pressure. The residue was dissolved in DMF (4 mL), then 4-(nitrooxy) butanoic acid, 2-1-3 (117.43 mg, 0.788 mmol, 1.1 equiv), HATU (408.36 mg, 1.074 mmol, 1.5 equiv) and DIEA (277.62 mg, 2.148 mmol, 3 equiv) was added into the solution. The mixture was stirred for one hour. The residue was purified by reversed-phase flash chromatography with the following conditions: column, C₁₈ silica gel; mobile phase, acetonitrile in water (0.05% NH₄HCO₃), 20% to 50% gradient in 12 min; detector, UV 220 nm. The eluent was lyophilized. This resulted in (R)-4-((1-(benzo[d][1,3]dioxol-5-yl) propan-2-yl)amino)-4-oxobutyl nitrate, 2-1 (25.7 mg) as a white solid. MS m/z [M+H]⁺ (ESI): 311.10. ¹H NMR (300 MHz, Chloroform-d) δ 6.79-6.50 (m, 3H), 5.93 (s, 2H), 5.26 (s, 1H), 4.60-4.31 (m, 2H), 4.31-4.08 (m, 1H), 2.76-2.55 (m, 2H), 2.28-2.16 (m, 2H), 2.15-1.92 (m, 2H), 1.12 (d, J=6.6 Hz, 3H).

### Plasma Stability of (R)-4-((1-(Benzo[ď] [1,3]Dioxol-5-Yl) Propan-2-Yl)Amino)-4-Oxobutyl Nitrate, Prodrug 2-1

TABLE 3
Stability of Prodrug 2-1 in Plasma of Different Species

| | | | Remaining Percentage (%) | | | | | |
|---|---|---|---|---|---|---|---|---|
| | | T_{1/2} | 0 | 15 | 30 | 60 | 120 | 120 |
| Compound | Species | (min) | min | min | min | min | min | min |
| Propantheline | Human | 14.78 | 100.00 | 72.22 | 39.13 | 6.46 | 0.12 | 0.12 |
| Prodrug 2-1 | | 908.64 | 100.00 | 100.94 | 102.03 | 90.66 | 93.79 | 93.79 |
| Lovastatin | Rat | 4.48 | 100.00 | 9.82 | 0.00 | 0.00 | 0.00 | 0.00 |
| Prodrug 2-1 | | 230.30 | 100.00 | 96.93 | 93.53 | 81.00 | 70.84 | 70.84 |
| Propantheline | Mouse | 26.96 | 100.00 | 72.20 | 49.21 | 22.77 | 4.69 | 4.69 |
| Prodrug 2-1 | | 1573.92 | 100.00 | 95.13 | 98.19 | 95.02 | 93.59 | 93.59 |

Propantheline and Lovastatin are reference compounds.

### Scheme 3

TABLE 4
Solubility Result of 2-1 and Control Compound in FaSSIF

| Compounds | Solublity (uM) FaSSIF |
|---|---|
| Diclofenace | 285.47 |
| 2-1 | 284.33 |

Diclofenace is a reference compound

TABLE 5
Stability results of test compound and control compound
in PBS pH 6.5, SGF with and without pepsin

| | | T_{1/2} | Remaining Percentage (%) | | | | |
|---|---|---|---|---|---|---|---|
| Compounds | Incubation | (min) | 0 min | 30 min | 60 min | 120 min | 240 min |
| Chlorambucil | PBS (pH 6.5) | 47.72 | 100 | 62.6 | 39.9 | 15.07 | 2.05 |
| Erythromycin | SGF with pepsin | 39.82 | 100 | 59.9 | 39.02 | 12.40 | 1.57 |
| Erythromycin | SGF without pepsin | 40.25 | 100 | 60.53 | 34.86 | 11.45 | 1.64 |
| 2-1 | PBS (pH 6.5) | 802.89 | 100 | 104.05 | 105.81 | 98.20 | 83.83 |
| 2-1 | SGF with pepsin | 811.92 | 100 | 98.88 | 98.96 | 89.53 | 82.75 |
| 2-1 | SGF without pepsin | 858.66 | 100 | 95.42 | 99.51 | 93.54 | 81.87 |

Chlorambucil and Erythromycin are reference compounds.

1. A compound of Formula (I): 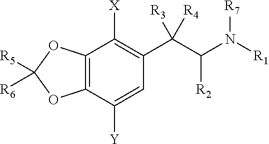
or a pharmaceutically acceptable salt thereof; wherein,
R₁ is —(C═O)(CR₉R₉′)ₙ—ONO₂ or —(C═O)(CR₉R₉′)ₘ—CH(NH₂)CH₂ONO₂;
R₂ is H, C₁-C₄ alkyl, or CF₃;
R₃ and R₄ are independently H or F;
R₅ and R₆ are independently H, C₁-C₆ alkyl, or CF₃;
R₇ is H or CH₃;
R₈ is H, C₁-C₆ alkyl, or CF₃;
R₉ and R₉′ are independently H, halogen, or C₁-C₆ alkyl;
X and Y are independently H, F, Cl, Br, or OR⁸,
n is an integer from 1 to 9; and
m is an integer from 1 to 9.

2. The compound of claim 1, wherein R₁ is —(C═O)(CR₉R₉′)ₙ—ONO₂.

3. The compound of claim 1, wherein R₁ is —(C═O)(CR₉R₉′)ₘ—CH(NH₂)CH₂ONO₂.

4. The compound of claim 1, wherein R₁ is —(C═O)(CH₂)ₘ—CH(NH₂)CH₂ONO₂.

5. The compound of claim 1, wherein R₂ is C₁-C₄ alkyl.

6. The compound of claim 1, wherein R₂ is methyl.

7. The compound of claim 1, wherein R₂ is CF₃.

8. The compound of claim 1, wherein R₃ and R₄ are H.

9. The compound of claim 1, wherein R₃ and R₄ are F.

10. The compound of claim 1, wherein R₅ and R₆ are H.

11. The compound of claim 1, wherein R₅ and R₆ are C₁-C₆ alkyl.

12. The compound of claim 1, wherein R₅ and R₆ are CF₃.

13. The compound of claim 1, wherein R₇ is H.

14. The compound of claim 1, wherein R₇ is CH₃.

15. The compound of claim 1, wherein X and Y are H.

16. The compound of claim 1, wherein X and Y are F.

17. The compound of claim 1, wherein m is 2.

18. The compound of claim 1, wherein n is 3.

19. The compound of claim 1, wherein n is 4.

20. The compound of claim 1, having the formula: 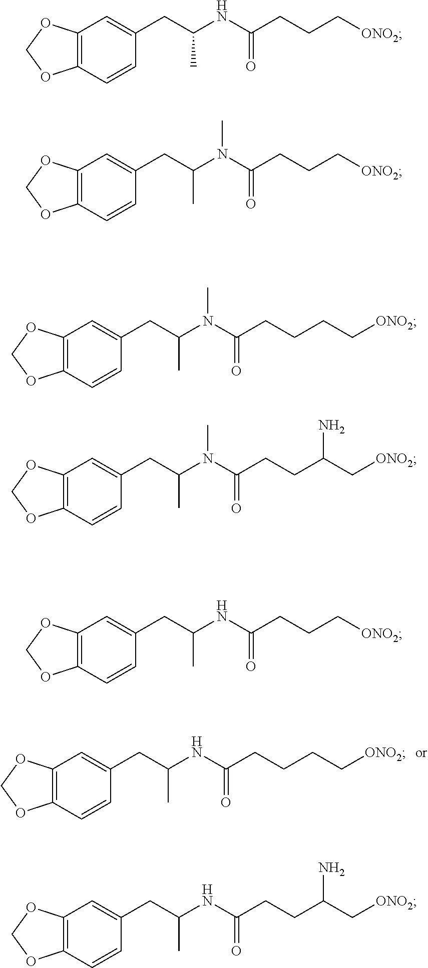
or a pharmaceutically acceptable salt thereof.

21. A compound of Formula (II): 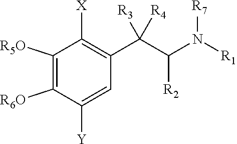
or a pharmaceutically acceptable salt thereof; wherein
R₁ is —(C═O)(CR₉R₉′)ₙ—ONO₂ or —(C═O)(CR₉R₉′)ₘ—CH(NH₂)CH₂ONO₂;
R₂ is H, C₁-C₄ alkyl or CF₃;
R₃ and R₄ are independently H or F;
R₅ and R₆ are independently H, C₁-C₆ alkyl, or CF₃;
R₇ is H or CH₃;
R₈ is H, C₁-C₆ alkyl, or CF;
R₉ and R₉′ are independently H, halogen, or C₁-C₆ alkyl;
X and Y are independently H, F, Cl, Br, or OR⁸,
n is an integer from 1 to 9; and
m is an integer from 1 to 9.

22. The compound of claim 21, wherein R₁ is —(C═O)(CR₉R₉′)ₙ—ONO₂.

23. The compound of claim 21, wherein R₁ is —(C═O)(CR₉R₉′)ₘ—CH(NH₂)CH₂ONO₂.

24. The compound of claim 21, wherein R₁ is —(C═O)(CH₂)ₙ—ONO₂.

25. The compound of claim 21, wherein R₁ is —(C═O)(CH₂)ₘ—CH(NH₂)CH₂ONO₂.

26. The compound of claim 21, wherein R₂ is C₁-C₄ alkyl.

27. The compound of claim 21, wherein R₂ is methyl.

28. The compound of claim 21, wherein R₂ is CF₃.

29. The compound of claim 21, wherein R₃ and R₄ are H.

30. The compound of claim 21, wherein R₃ and R₄ are F.

31. The compound of claim 21, wherein R₅ and R₆ are H.

32. The compound of claim 21, wherein R₅ and R₆ are C₁-C₆ alkyl.

33. The compound of claim 21, wherein R₅ and R₆ are CF₃.

34. The compound of claim 21, wherein R₇ is H.

35. The compound of claim 21, wherein R₇ is CH₃.

36. The compound of claim 21, wherein X and Y are H.

37. The compound of claim 21, wherein X and Y are F.

38. The compound of claim 21, wherein m is 2.

39. The compound of claim 21, wherein n is 3.

40. The compound of claim 21, wherein n is 4.

41. A compound of Formula (III): 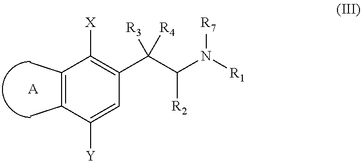
or a pharmaceutically acceptable salt thereof; wherein,
A is 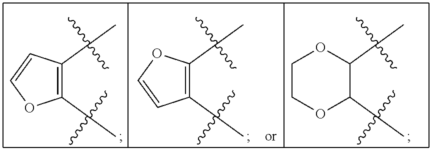 [SMILES: *c1ccoc1* ; *c1ccoc1* ; *C1OCCOC1*]
R₁ is —(C═O)(CR₉R₉′)ₙ—ONO₂ or —(C═O)(CR₉R₉′)ₘ—CH(NH₂)CH₂ONO₂,
R₂ is H, C₁-C₄ alkyl or CF₃;
R₃ and R₄ are independently H or F;
R₅ and R₆ are independently H, C₁-C₆ alkyl, or CF₃;
R₇ is H or CH₃; and
R₈ is H, C₁-C₆ alkyl, or CF₃
R₉ and R₉′ are independently H, halogen, or C₁-C₆ alkyl;
X and Y are independently H, F, Cl, Br, or OR⁸,
n is an integer from 1 to 9; and
m is an integer from 1 to 9.

42. The compound of claim 41, wherein R₁ is —(C═O)(CR₉R₉′)ₙ—ONO₂.

43. The compound of claim 41, wherein R₁ is —(C═O)(CR₉R₉′)ₘ—CH(NH₂)CH₂ONO₂.

44. The compound of claim 41, wherein R₁ is —(C═O)(CH₂)ₙ—ONO₂.

45. The compound of claim 41, wherein R₁ is —(C═O)(CH₂)ₘ—CH(NH₂)CH₂ONO₂.

46. The compound of claim 41, wherein R₂ is C₁-C₄ alkyl.

47. The compound of claim 41, wherein R₂ is methyl.

48. The compound of claim 41, wherein R₂ is CF₃.

49. The compound of claim 41, wherein R₃ and R₄ are H.

50. The compound of claim 41, wherein R₃ and R₄ are F.

51. The compound of claim 41, wherein R₇ is H.

52. The compound of claim 41, wherein R₇ is CH₃.

53. The compound of claim 41, wherein X and Y are H.

54. The compound of claim 41, wherein X and Y are F.

55. The compound of claim 41, wherein m is 2.

56. The compound of claim 41, wherein n is 3.

57. The compound of claim 41, wherein n is 4.

58. The compound of claim 41, wherein A is

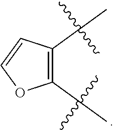 [SMILES: *c1ccoc1*]

59. The compound of claim 41, wherein A is

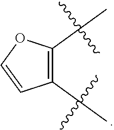 [SMILES: *c1ccoc1*]

60. The compound of claim 41, wherein A is

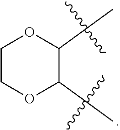 [SMILES: *C1OCCOC1*]

61. A compound having the formula: 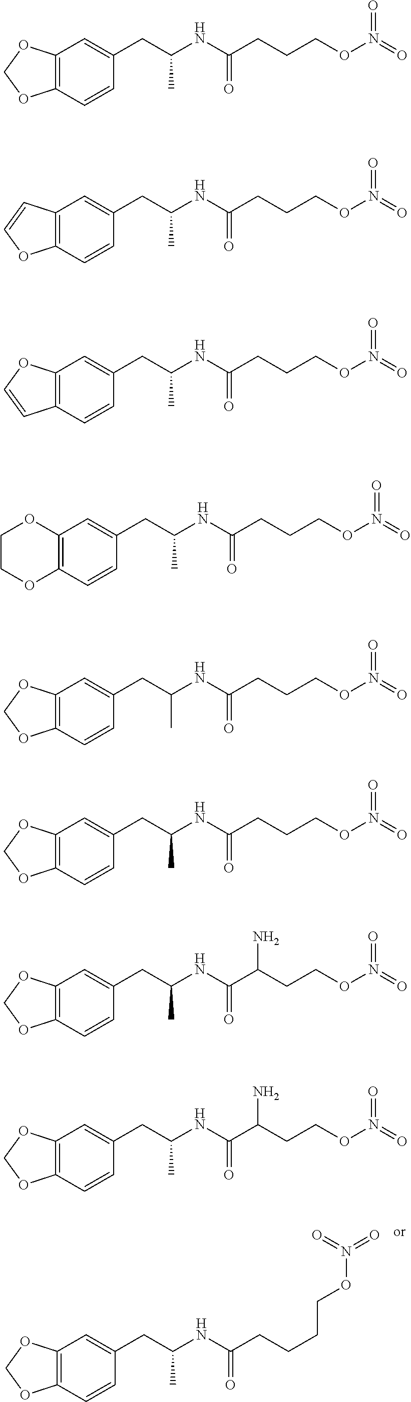 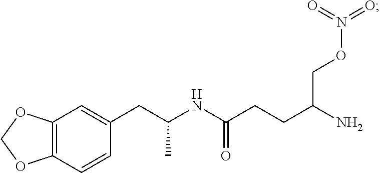 [SMILES: C[C@H](Cc1ccc2c(c1)OCO2)NC(=O)CCC(N)CO[N+](=O)[O-]]
or a pharmaceutically acceptable salt thereof.

62. A pharmaceutical composition, comprising a compound of claim 1 and a pharmaceutically acceptable excipient.

63. A method of treating social anxiety, the method comprising administering a therapeutically effective amount of a compound of claim 1.
